SEQUENCE LISTING (15 sequences)

SEQ ID NO: 1 - protein, 108 aa, Artificial Sequence
  Description of Artificial Sequence Synthetic polypeptide
QDIQMTQSPSFLSASVGDRVTISCRASQYISIYLNWYQQRPGEAPKLLINAASSLQSGDPSRFSGSGSGTDFTLTINSLQPDDFATYYCQQYKSYPLTFGEGTKVEIK

SEQ ID NO: 2 - DNA, 324 bp, Artificial Sequence
  Description of Artificial Sequence Synthetic polynucleotide
caagacatccagatgacccagtctccatccttcctgtccgcatctgtaggagacagagtcaccatctcttgccgggcaagtcagtacatcagcatatatttgaattggtatcagcagagaccaggggaagcccctaaactcctgatcaatgctgcatccagtttgcaaagtggggacccatcaaggttcagtggcagtggatctgggacagatttcactctcaccatcaacagcctgcagcctgatgattttgcaacttattactgccaacagtataagagttaccccctcactttcggcgaggggaccaaggtggagatcaaa

SEQ ID NO: 3 - protein, 118 aa, Artificial Sequence
  Description of Artificial Sequence Synthetic polypeptide
EVQLLESGGGLVQPGGSLRLSCAASGFTFSAYGMGWVRQAPGKGLEWVSVISPSGGQTSYADSVKGRFTISRDNSKNTLYLQMNSLRAEDTALYYCAGGDRYGPLHYWGQGTLVTVSS

SEQ ID NO: 4 - DNA, 354 bp, Artificial Sequence
  Description of Artificial Sequence Synthetic polynucleotide
gaagttcaattgttagagtctggtggcggtcttgttcagcctggtggttctttacgtctttcttgcgctgcttccggattcactttctctgcttacggtatgggttgggttcgccaagctcctggtaaaggtttggagtgggtttctgttatctctccttctggtggccagacttcttatgctgactccgttaaaggtcgcttcactatctctagagacaactctaagaatactctctacttgcagatgaacagcttaagggctgaggacaccgccttgtattactgtgcgggaggggacaggtatggacccttgcactactggggccagggaaccctggtcaccgtctcaagc

SEQ ID NO: 5 - protein, 214 aa, Homo sapiens
DIQMTQSPSSLSASVGDRVTITCRASQGIGHYLAWYQQKPGKVPKLLIYTASTLQSGVPSRFSGSGSGTDFTLTISSLQPEDVATYYCQQFNSYPHTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 450 aa, Homo sapiens
EVQLLESGGGLVQPGGSLRLSCAASGFTFSMYGMVWVRQAPGKGLEWVSVISPSGGNTGYADSVKGRFTISRDNSKNTLYLQMNSLRAEDTAVYYCARAPRGYSYGYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 660 bp, Homo sapiens
ggcgtgcactctgacatccagatgacccagtctccatcctccctgtctgcatctgtaggagacagagtcaccatcacttgccgggcgagtcagggcattggccattatttagcctggtatcagcagaaaccagggaaagttcctaagctcctgatctatactgcatccactttgcaatcaggggtcccatctcggttcagtggcagtggatctgggacagatttcactctcaccatcagcagcctgcagcctgaagatgttgcaacttattactgtcaacagtttaatagttaccctcacaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 8 - DNA, 1353 bp, Homo sapiens
gaagttcaattgttagagtctggtggcggtcttgttcagcctggtggttctttacgtctttcttgcgctgcttccggattcactttctctatgtacggtatggtttgggttcgccaagctcctggtaaaggtttggagtgggtttctgttatctctccttctggtggcaatactggttatgctgactccgttaaaggtcgcttcactatctctagagacaactctaagaatactctctacttgcagatgaacagcttaagggctgaggacactgcagtctactattgtgcgagagccccacgtggatacagctatggttactactactggggccagggaaccctggtcaccgtctcaagcgcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctccggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 9 - protein, 107 aa, Homo sapiens
DIQMTQSPSSLSASVGDRVTITCRASQGIGHYLAWYQQKPGKVPKLLIYTASTLQSGVPSRFSGSGSGTDFTLTISSLQPEDVATYYCQQFNSYPHTFGQGTRLEIK

SEQ ID NO: 10 - protein, 107 aa, Homo sapiens [sequence omitted]

SEQ ID NO: 11 - protein, 120 aa, Homo sapiens
EVQLLESGGGLVQPGGSLRLSCAASGFTFSMYGMVWVRQAPGKGLEWVSVISPSGGNTGYADSVKGRFTISRDNSKNTLYLQMNSLRAEDTAVYYCARAPRGYSYGYYYWGQGTLVTVSS

SEQ ID NO: 12 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 19 aa, Artificial Sequence
  Description of Artificial Sequence Synthetic peptide
MGWSCIILFLVATATGVHS

SEQ ID NO: 14 - protein, 19 aa, Artificial Sequence
  Description of Artificial Sequence Synthetic peptide
MGWSCIILFLVATATGAHS

SEQ ID NO: 15 - protein, 1138 aa, Artificial Sequence
  Description of Artificial Sequence Synthetic construct
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRCEQACREGRFGQSCQEQCPGISGCRGLTFCLPDPYGCSCGSGWRGSQCQEACAPGHFGADCRLQCQCQNGGTCDRFSGCVCPSGWHGVHCEKSDRIPQILNMASELEFNLETMPRINCAAAGNPFPVRGSIELRKPDGTVLLSTKAIVEPEKTTAEFEVPRLVLADSGFWECRVSTSGGQDSRRFKVNVKVPPVPLAAPRLLTKQSRQLVVSPLVSFSGDGPISTVRLHYRPQDSTMDWSTIVVDPSENVTLMNLRPKTGYSVRVQLSRPGEGGEGAWGPPTLMTTDCPEPLLQPWLEGWHVEGTDRLRVSWSLPLVPGPLVGDGFLLRLWDGTRGQERRENVSSPQARTALLTGLTPGTHYQLDVQLYHCTLLGPASPPAHVLLPPSGPPAPRHLHAQALSDSEIQLTWKHPEALPGPISKYVVEVQVAGGAGDPLWIDVDRPEETSTIIRGLNASTRYLFRMRASIQGLGDWSNTVEESTLGNGLQAEGPVQESRAAEEGLDQQLILAVVGSVSATCLTILAALLTLVCIRRSCLHRRRTFTYQSGSGEETILQFSSGTLTLTRRPKLQPEPLSYPVLEWEDITFEDLIGEGNFGQVIRAMIKKDGLKMNAAIKMLKEYASENDHRDFAGELEVLCKLGHHPNIINLLGACKNRGYLYIAIEYAPYGNLLDFLRKSRVLETDPAFAREHGTASTLSSRQLLRFASDAANGMQYLSEKQFIHRDLAARNVLVGENLASKIADFGLSRGEEVYVKKTMGRLPVRWMAIESLNYSVYTTKSDVWSFGVLLWEIVSLGGTPYCGMTCAELYEKLPQGYRMEQPRNCDDEVYELMRQCWRDRPYERPPFAQIALQLGRMLEARKAYVNMSLFENFTYAGIDATAEEA

(19) United States
(12) Patent Application Publication
(10) Pub. No.: US 2008/0213253 A1
(43) Pub. Date: Sep. 4, 2008

(54) COMBINATION THERAPY FOR THE TREATMENT OF CANCER

(75) Inventors: Clive R. Wood, Boston, MA (US); Daniel T. Dransfield, Hanson, MA (US)

Correspondence Address: LOWRIE, LANDO & ANASTASI, LLP, ONE MAIN STREET, SUITE 1100, CAMBRIDGE, MA, 02142, US

(73) Assignee: DYAX CORP., Cambridge, MA (US)

(21) Appl. No.: 11/972,655

(22) Filed: Jan. 11, 2008

Related U.S. Application Data
(60) Provisional application No. 60/884,702, filed on Jan. 12, 2007.

(51) Int. Cl. A61K 39/395; A61K 38/17; A61P 9/00

(52) U.S. Cl. 424/130.1; 514/2

(57) ABSTRACT

Disclosed are new methods for treatment of angiogenesis-related disorders. Angiogenesis-related disorders are treated by administration of a Tie1 ectodomain-binding agent and a vascular disrupting agent.

### CROSS REFERENCE TO RELATED APPLICATIONS

This application claims priority to U.S. Application Ser. No. 60/884,702, filed on Jan. 12, 2007, the contents of which are hereby incorporated by reference in its entirety.

### BACKGROUND

Blood vessels are composed of an inner layer of endothelial cells and an outer layer of pericytes or smooth muscle cells. The first tubular structures are formed by endothelial cells that subsequently recruit pericytes and smooth muscle cells to ensheath them. The de novo formation of blood vessels from a dispersed population of mesodermally derived endothelial precursor cells is termed vasculogenesis. This primitive network undergoes successive morphogenetic events including sprouting, splitting, and remodeling to generate the hierarchical vascular network from large to branched small vessels. These successive morphogenetic events are collectively called angiogenesis.

Tie1 and Tie2 are receptor tyrosine kinases (RTKs) that are expressed almost exclusively in endothelial cells and hematopoietic precursor cells. These two receptors are required for the normal development of vascular structures during embryogenesis. The two Tie receptors form a RTK subfamily since, unlike other RTK family members, they include extracellular EGF-homology domains. See, e.g., Partanen (1992) Mol. Cell Biol. 12:1698 and WO 93/14124. Targeted disruption of the Tie1 gene in mice results in a lethal phenotype characterized by extensive hemorrhage and defective microvessel integrity. See, e.g., Puri et al. (1995) EMBO J. 14:5884. Tie2 null embryos have defects in vascular remodeling and maturation, resulting from improper recruitment of periendothelial supporting cells. Angiopoietins (Ang, e.g., Ang1, Ang2, Ang3, and Ang4) are proteins that interact with Tie2.

### SUMMARY

In one aspect, the invention provides methods for treating (e.g., ameliorating at least one symptom of) an angiogenesis-related disorder by administering a Tie1 ectodomain-binding agent and a vascular disrupting agent (VDA).

In one embodiment, the Tie1 ectodomain-binding agent is administered for a period prior to the administration of the VDA. The period of Tie1 ectodomain-binding agent administration may be less than one day (e.g., less than one hour, or about 1, 2, 3, 4, 6, 8, 12, 18, or 24 hours) or range from 1 day up to 35 days (e.g., 1, 2, 3, 4, 5, 6, 7, 8, 10, 12, 14, 20, 21, 28, 30, or 35 days, or any day or range in between) prior to the first administration of the VDA. The period of Tie1 ectodomain-binding protein administration may be followed by a hiatus period during which neither the Tie1 ectodomain-binding protein nor the VDA are administered. The hiatus period may be may be less than one day (e.g., less than one hour, or about 1, 2, 3, 4, 6, 8, 12, 18, or 24 hours) or range from 1 day up to 35 days (e.g., 1, 2, 3, 4, 5, 6, 7, 8, 10, 12, 14, 20, 21, 28, 30, or 35 days, or any day or range in between) prior to the first administration of the VDA.

In another embodiment, the Tie1 ectodomain-binding agent is administered following first administration of the VDA. The period between first administration of the VDA and administration of the Tie1 ectodomain-binding agent may be less than one day (e.g., less than one hour, or about 1, 2, 3, 4, 6, 8, 12, 18, or 24 hours) or range from 1 day up to 35 days (e.g., 1, 2, 3, 4, 5, 6, 7, 8, 10, 12, 14, 20, 21, 28, 30, or 35 days, or any day or range in between).

In some embodiments, administration of the Tie1 ectodomain-binding agent is continued following the first administration of the VDA, while in other embodiments, the administration of the Tie1 ectodomain-binding agent is discontinued upon initiation of VDA administration. In some embodiments in which administration of the Tie1 ectodomain-binding agent is discontinued following initiation of administration of the VDA, administration of the Tie1 ectodomain-binding agent is not re-initiated.

In some embodiments, the combination therapy disclosed herein may be administered in a series (two or more) of cycles. For example, in configurations in which the Tie1 ectodomain-binding agent is administered for a period prior to the initiation of administration of the VDA and the Tie1 ectodomain-binding agent is discontinued upon, or following, the initiation of VDA administration, the Tie1 ectodomain-binding agent may be reinitiated following administration or completion of administration of the VDA.

In some embodiments, the combination therapy disclosed herein utilizes a schedule of alternating agents. For example, the Tie1 ectodomain-binding agent is administered for a period, followed by administration of a VDA, followed by further administration of the Tie1 ectodomain-binding agent, followed by another administration of the VDA, etc. The converse schedule may also be used (VDA, then Tie1 ectodomain-binding agent, then VDA, then Tie1 ectodomain-binding agent, etc.). In some embodiments, the administration of one agent is discontinued prior to the administration of the other agent.

In some embodiments, the Tie1 ectodomain-binding agent and the VDA are each administered in an amount effective to individually ameliorate at least one symptom of an angiogenesis-related disorder in the subject or otherwise treat or prevent the disorder in a subject. In other embodiments, the Tie1 ectodomain-binding agent and the VDA are each administered in an amount that is less than an amount that is individually effective to ameliorate at least one symptom of an angiogenesis-related disorder in the subject or otherwise treat or prevent the disorder in a subject. In some embodiments, the VDA is administered in an amount that is less than an amount that is individually effective to ameliorate at least one symptom of an angiogenesis-related disorder in the subject or otherwise treat or prevent the disorder in a subject. In some embodiments, the Tie1 ectodomain-binding agent is administered in an amount that is less than an amount that is individually effective to ameliorate at least one symptom of an angiogenesis-related disorder in the subject or otherwise treat or prevent the disorder in a subject. In some embodiments, the Tie1 ectodomain-binding agent and the VDA are administered in synergistically effective amounts (e.g., amounts which, when compared to either compound administered alone, result in a synergistic effect).

Angiogenesis-related disorders include, but are not limited to, neoplastic disease (e.g., solid tumors, tumor metastases, and benign tumors, particularly neoplastic disease requiring a blood supply or angiogenesis); inflammatory disorders (e.g., rheumatoid arthritis, lupus, restenosis, psoriasis, graft v. host response, or multiple sclerosis); ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation.

Benign tumors include, but are not limited to hemangiomas, acoustic neuromas, neurofibromas, trachomas, and pyogenic granulomas. Solid tumors include, but are not limited to malignancies, e.g., sarcomas, adenocarcinomas, and carcinomas, of the various organ systems, such as those affecting lung, breast, gastrointestinal (e.g., colon), and genitourinary tract (e.g., renal, urothelial cells), pharynx, as well as adenocarcinomas which include malignancies such as most colon cancers, rectal cancer, renal-cell carcinoma, liver cancer, non-small cell carcinoma of the lung, cancer of the small intestine, cancer of the esophagus, and pancreatic cancer.

In some embodiments, the angiogenesis-related disorder is an inflammatory disorder, e.g., rheumatoid arthritis, psoriasis, rheumatoid or rheumatic inflammatory disease, or other chronic inflammatory disorders, such as chronic asthma, arterial or post-transplantational atherosclerosis, and endometriosis. Other angiogenesis-related disorders that can be treated include those that have deregulated or undesired angiogenesis, such as ocular neovascularization, e.g., retinopathies (including diabetic retinopathy and age-related macular degeneration) hemangioblastoma, hemangioma, and arteriosclerosis.

In one embodiment, the subject is in need of reduced angiogenesis, or identified as such. For example, the subject has (e.g., has been diagnosed with) a neoplastic disorder, e.g., a metastatic cancer. For example, the subject has an angiogenesis-dependent cancer or tumor. The tumor can be a solid tumor, e.g., a tumor at least 1, 2, 3, 5, 8 or 10 mm in diameter. In one embodiment, the solid tumor has a hypoxic core. The method can include, prior to administering the Tie1 ectodomain-binding agent and/or VDA, evaluating the subject and detecting a solid tumor in the subject.

In some embodiments, the Tie1 ectodomain-binding agent increases Tie complex formation. In some embodiments the Tie1 ectodomain-binding agent increases tyrosine phosphorylation of Tie1. In some embodiments, the Tie1 ectodomain-binding agent induces down modulation of Tie1 and Tie1/Tie2 complex from the surface of the cell.

In some embodiments, the Tie1 ectodomain-binding agent is an antibody or antigen binding fragment thereof that includes at least one complementarity determining region (CDR, e.g., HC CDR1, HC CDR2, HC CDR3, LC CDR1, LC CDR2, and/or LC CDR3) from E3 (DX-2240), E3b (DX-2220), M0044-A06; M0044-A11; M0044-B04; M0044-B05; M0044-B08; M0044-B09; M0044-B10; M0044-B12; M0044-C07; M0044-D01; M0044-E03; M0044-F03; M0044-F06; M0044-F09; M0044-G06; M0044-G07; M0044-G11; M0044-H03; M0044-H05; M0044-H07; M0044-H09; M0045-A02; M0045-A04; M0045-B01; M0045-B03; M0045-B11; M0045-C02; M0045-C11; M0045-C12; M0045-D01; M0045-D07; M0045-G01; M0045-G10; M0046-A11; M0046-B06; M0046-B10; M0046-G12; M0046-H03; M0046-H10; M0046-H11; M0047-B03; M0047-D01; M0047-D03; M0047-E10; M0047-G09; M0053-A02; M0053-A03; M0053-A05; M0053-A09; M0053-B09; M0053-B11; M0053-D03; M0053-D06; M0053-D12; M0053-E03; M0053-E04; M0053-E08; M0053-F04; M0053-F05; M0053-F06; M0053-F08; M0053-G04; M0053-G05; M0054-A08; M0054-B06; M0054-B08; M0054-C03; M0054-C07; M0054-E04; M0054-G01; M0054-G05; M0054-H10; M0055-A09; M0055-B11; M0055-B12; M0055-C05; M0055-C07; M0055-D03; M0055-D06; M0055-D12; M0055-E04; M0055-E06; M0055-E10; M0055-E12; M0055-F10; M0055-G02; M0055-G03; M0055-H04; M0056-A01; M0056-A06; M0056-B08; M0056-B09; M0056-C03; M0056-C04; M0056-E08; M0056-F01; M0056-F02; M0056-F10; M0056-F11; M0056-G03; M0056-G04; M0056-G08; M0056-G12; M0056-H04; M0056-H12; M0057-B05; M0057-H07; M0058-A09; M0058-D04; M0058-E09; M0058-F03; M0058-G03; M0058-H01; M0059-A02; M0059-A06; M0060-B02; M0060-H01; M0061-A03; M0061-C05; M0061-C06; M0061-F07; M0061-G12; M0061-H09; M0062-A12; M0062-B05; M0062-B07; M0062-C08; M0062-D04; M0062-E02; M0062-E03; M0062-E11; M0062-F10; M0062-G06; or M0062-H01. In some embodiments, the antibody or antigen binding fragment thereof includes 2, 3, 4, 5 or 6 CDRs from the aforementioned antibodies.

A VDA is an agent that disrupts existing tumor vasculature. Examples of VDAs include combretastatin-related compounds (e.g., combretastatin A-4 disodium phosphate (CA4P), combretastatin prodrugs (e.g., AVE8062 and OXI4503)), colchicine-related compounds (e.g., ZD6126), flavone-related compounds (e.g., flavone acetic acid and AS1404), dolastatin 10 derivatives (e.g., TZT1027), other microtubule disrupting agents such as MPC-6827 (Myriad Genetics), CYT997 (Cytopia Ltd.), and BNC105 (Bionomics Ltd.).

In some embodiments, the administration of a Tie1 ectodomain-binding agent and a VDA is used as an adjuvant therapy. The adjuvant therapy can be a post-operative therapy that is administered to the subject after the subject has undergone surgery to remove all or part of a tumor (e.g., after surgery to treat glioblastoma or colorectal, breast, or lung cancer). In some embodiments, administration in accordance with the invention is initiated within 6, 12, 24, 48, or 100 hours of surgery. The adjuvant therapy can be a pre-operative (or pre-radiotherapy or pre-chemotherapy) neoadjuvant therapy. In some embodiments, administration in accordance with the invention is initiated 1, 2, 3, 4, 5, 7, 10, 14, 18, 21, 24, or 28 days or one month prior to the surgical, radiation, or chemotherapy.

In some embodiments, the method includes an additional therapeutic modality. For example, the additional therapeutic modality is radiation therapy, an anti-angiogenic therapy (e.g., a VEGF pathway antagonist such as VEGF trap or an anti-VEGF antibody such as bevacizumab or ranibizumab), or a cytotoxic chemotherapy agent such as an anti-metabolite (e.g., 5-FU, with leucovorin), irinotecan, (or other topoisomerase inhibitor), doxorubicin, or any combination all of these agents, including administration of all of these agents.

The methods can further include the step of monitoring the subject, e.g., for a reduction in one or more of: a reduction in tumor size; reduction in cancer markers, e.g., levels of cancer specific antigen; reduction in the appearance of new lesions, e.g., in a bone scan; a reduction in the appearance of new disease-related symptoms; or decreased or stabilization of size of soft tissue mass; or any parameter related to improvement in clinical outcome. The subject can be monitored in one or more of the following periods: prior to beginning of treatment; during the treatment; or after one or more elements of the treatment have been administered. Monitoring can be used to evaluate the need for further treatment with the same Tie1-binding protein and/or VDA or for additional treatment with additional agents. Generally, a decrease in or stabilization of one or more of the parameters described above is indicative of the improved condition of the subject. Information about the monitoring can be recorded, e.g., in electronic or digital form.

The subject can be a mammal, e.g., a primate, preferably a higher primate, e.g., a human.

In another aspect, the invention includes the use of a Tie1 ectodomain-binding agent and a VDA for the manufacture of a medicament for treating an angiogenesis-related disorder in accordance with the methods disclosed herein.

Other features and advantages of the instant invention will become more apparent from the following detailed description and claims. Embodiments of the invention can include any combination of features described herein. In no case does the term “embodiment” necessarily exclude one or more other features disclosed herein, e.g., in another embodiment. The contents of all references, patent applications and patents, cited throughout this application are hereby expressly incorporated by reference.

### DETAILED DESCRIPTION

Disclosed herein are new methods for treating angiogenesis-related disorders by administering a Tie1 ectodomain-binding agent and a vascular disrupting agent (VDA).

The term “treat” or “treatment” refers to the application or administration of an agent, alone or in combination with one or more other agents (e.g., a second agent) to a subject, e.g., a patient, e.g., a patient who has a disorder (e.g., a disorder as described herein), a symptom of a disorder or a predisposition for a disorder, e.g., to cure, heal, alleviate, relieve, alter, remedy, ameliorate, improve or affect the disorder, the symptoms of the disorder or the predisposition toward the disorder. Treating a cell refers to a reduction in an activity of a cell, e.g., ability of an endothelial cell to form tubes or vessels. A reduction does not necessarily require a total elimination of activity, but a reduction, e.g., a statistically significant reduction, in the activity or the number of the cell.

As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂, a Fd fragment, a Fv fragments, and dAb fragments) as well as complete antibodies.

The VH and VL regions can be further subdivided into regions of hypervariability, termed “complementarity determining regions” (CDR), interspersed with regions that are more conserved, termed “framework regions” (FR). The extent of the framework regions and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay 1988 Ann. Rev Immunol. 6:381-405). The canonical structures of hypervariable loops of an immunoglobulin variable can be inferred from its sequence, as described in Chothia et al. (1992) J. Mol. Biol. 227:799-817; Tomlinson et al. (1992) J. Mol. Biol. 227:776-798); and Tomlinson et al. (1995) EMBO J. 14(18):4628-38.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with Tie1, e.g., binds to, activates, or inhibits Tie1.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region includes three domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The term “antibody” includes intact immunoglobulins of types IgA, IgG, IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity.

The term “monospecific antibody” refers to an antibody that displays a single binding specificity and affinity for a particular target, e.g., epitope. This term includes a “monoclonal antibody” which refers to an antibody that is produced as a single molecular species, e.g., from a population of homogenous isolated cells. A “monoclonal antibody composition” refers to a preparation of antibodies or fragments thereof of in a composition that includes a single molecular species of antibody. In one embodiment, a monoclonal antibody is produced by a mammalian cell. One or more monoclonal antibody species may be combined.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human or effectively human. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human or effectively human. For example, FR1, FR2, and FR3 collectively can be at least 70, 75, 80, 85, 90, 92, 95, 98, or 99% identical to a human sequence encoded by a human germline V segment of a locus encoding a light or heavy chain sequence.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin light chains (about 25 kDa or 214 amino acids) are encoded by a variable region gene at the NH₂-terminus (about 110 amino acids) and a kappa or lambda constant region gene at the COOH-terminus. Full-length immunoglobulin heavy chains (about 50 kDa or 446 amino acids), are similarly encoded by a variable region gene (about 116 amino acids) and one of the other aforementioned constant region genes, e.g., gamma (encoding about 330 amino acids). A light chain refers to any polypeptide that includes a light chain variable domain. A heavy chain refers to any polypeptide that includes a heavy chain variable domain.

The term “antigen-binding fragment” of a full-length antibody (or simply “antibody portion,” or “fragment”), as used herein, refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Antibody fragments can be obtained using any appropriate technique including conventional techniques known to those with skill in the art. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition. As used herein, “isotype” refers to the antibody class (e.g., IgM or IgG1) that is encoded by heavy chain constant region genes.

In one embodiment, the HC or LC of an antibody includes sequences that correspond to an amino acid sequence encoded by a human germline sequence, e.g., the framework regions and/or in the CDRs. For example, the antibody can include framework sequences from the human germline antibody sequence DP47 (3-23). In one embodiment, one or more codons for the antibody are altered relative to the germline nucleic acid sequence, but are chosen to encode the same amino acid sequence. Codons can be selected, e.g., to optimize expression in a particular system, create restriction enzyme sites, create a silent fingerprint, etc.

A “humanized” immunoglobulin variable region is an immunoglobulin variable region that includes a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

As used herein, “Tie complex” refers to either a heteromeric complex that includes Tie1 and Tie2 (and may include an angiopoietin (Ang)) or a homomeric complex of Tie1. The heteromeric Tie complex is formed in part by association of the extracellular and/or intracellular domains of Tie1 and Tie2 and may also include Ang. As used herein, “complex members” refers to the proteins that are included in a heteromeric Tie complex. Accordingly, Tie1 and Tie2, and optionally Ang, are all complex members. The term “Ang” includes all angiopoietins, such as Ang1, Ang2, Ang3, and Ang4. The heteromeric Tie complex can include other proteins in addition to Tie1, Tie2, and Ang.

“Angiogenesis” includes all stages of vessel development (e.g., blood or lymphatic vessel development), including initial vessel formation and later vessel remodeling and morphological changes.

As used herein, the terms “agonist” and “antagonist” describe properties in context of a particular activity or effect. For example, the E3 or E3b antibody can be an agonist in the context of promoting Tie1 self-association (e.g., homodimerization), yet an antagonist in the context of decreasing or inhibiting Tie complex formation and tube formation by human umbilical vein endothelial cells (HUVECs). Likewise, an agent that is an agonist in the context of a Tie1 signaling pathway can be an antagonist in the context of endothelial cell sprouting, splitting, and tube formation.

The term “Tie1 ectodomain” refers to an extracellular region of a Tie1 protein, e.g., a region that includes about amino acids 25-759 of SEQ ID NO:100 (see also SEQ ID NO:2 of US 2006/0057138):

(SEQ ID NO:100)

MVWRVPPFLLPILFLASHVGAAVDLTLLANLRLTDPQRFFLTCVSGEAGA
GRGSDAWGPPLLLEKDDRIVRTPPGPPLRLARNGSHQVTLRGFSKPSDLV
GVFSCVGGAGARRTRVIYVHNSPGAHLLPDKVTHTVNKGDTAVLSARVHK
EKQTDVIWKSNGSYFYTLDWHEAQDGRFLLQLPNVQPPSSGIYSATYLEA
SPLGSAFFRLIVRGCGAGRWGPGCTKECPGCLHGGVCHDHDGECVCPPGF
TGTRCEQACREGRFGQSCQEQCPGISGCRGLTFCLPDPYGCSCGSGWRGS
QCQEACAPGHFGADCRLQCQCQNGGTCDRFSGCVCPSGWHGVHCEKSDRI
PQILNMASELEFNLETMPRINCAAAGNPFPVRGSIELRKPDGTVLLSTKA
IVEPEKTTAEFEVPRLVLADSGFWECRVSTSGGQDSRRFKVNVKVPPVPL
AAPRLLTKQSRQLVVSPLVSFSGDGPISTVRLHYRPQDSTMDWSTIVVDP
SENVTLMNLRPKTGYSVRVQLSRPGEGGEGAWGPPTLMTTDCPEPLLQPW
LEGWHVEGTDRLRVSWSLPLVPGPLVGDGFLLRLWDGTRGQERRENVSSP
QARTALLTGLTPGTHYQLDVQLYHCTLLGPASPPAHVLLPPSGPPAPRHL
HAQALSDSEIQLTWKHPEALPGPISKYVVEVQVAGGAGDPLWIDVDRPEE
TSTIIRGLNASTRYLFRMRASIQGLGDWSNTVEESTLGNGLQAEGPVQES
RAAEEGLDQQLILAVVGSVSATCLTILAALLTLVCIRRSCLHRRRTFTYQ
SGSGEETILQFSSGTLTLTRRPKLQPEPLSYPVLEWEDITFEDLIGEGNF
GQVIRAMIKKDGLKMNAAIKMLKEYASENDHRDFAGELEVLCKLGHHPNI
INLLGACKNRGYLYIAIEYAPYGNLLDFLRKSRVLETDPAFAREHGTAST
LSSRQLLRFASDAANGMQYLSEKQFIHRDLAARNVLVGENLASKIADFGL
SRGEEVYVKKTMGRLPVRWMAIESLNYSVYTTKSDVWSFGVLLWEIVSLG
GTPYCGMTCAELYEKLPQGYRMEQPRNCDDEVYELMRQCWRDRPYERPPF
AQIALQLGRMLEARKAYVNMSLFENFTYAGIDATAEEA

Other exemplary regions are regions that include one or more EGF-like domains (e.g., 214-256, 258-303, 303-345, 214-303, 258-345, or 214-345 of SEQ ID NO:100); one or more Ig-Like C2-type domains (e.g., 43-105, 43-426, 372-426); one or more Fibronectin Type III repeats (e.g., 446-540, 543-639, 643-744, 446-639, 543-744, or 446-744 of SEQ ID NO:100); and combinations thereof. The terms “first Ig-like C2-type domain” and “Ig 1” refer to the immunoglobulin-like domain in Tie1 or Tie2 that is located closest to the amino terminus of the protein relative to the other Ig-like C2-type domain (the second such domain). For example, for Tie1, the first Immunoglobulin-like C2-type domain is located at about residue 43 to about residue 105 and the second Ig-like C2-type domain is located at about residue 372 to about residue 426.

As used herein, “binding affinity” refers to the apparent association constant or Kₐ. The Kₐ is the reciprocal of the dissociation constant (K_{d}). A ligand may, for example, have a binding affinity of at least 10⁵, 10⁶, 10⁷ or 10⁸ M⁻¹ for a particular target molecule. Higher affinity binding of a ligand to a first target relative to a second target can be indicated by a higher Kₐ (or a smaller numerical value K_{d}) for binding the first target than the Kₐ (or numerical value K_{d}) for binding the second target. In such cases the ligand has specificity for the first target relative to the second target. Differences in binding affinity (e.g., for specificity or other comparisons) can be at least 1.5, 2, 5, 10, 50, 100, or 1000-fold. For example, a Tie1 ectodomain-binding agent may preferentially bind to Tie1 at least 1.5, 2, 5, 10, 50, 100, or 1000-fold better than to another antigen, e.g., Tie2, EGF, fibronectin, or human serum albumin.

Binding affinity can be determined by a variety of methods including equilibrium dialysis, equilibrium binding, gel filtration, ELISA, surface plasmon resonance, or spectroscopy (e.g., using a fluorescence assay). These techniques can be used to measure the concentration of bound and free ligand as a function of ligand (or target) concentration. The concentration of bound ligand ([Bound]) is related to the concentration of free ligand ([Free]) and the concentration of binding sites for the ligand on the target where (N) is the number of binding sites per target molecule by the following equation:

[Bound]=N·[Free]/((1/Kₐ)+[Free])

Although quantitative measurements of Kₐ are routine, it is not always necessary to make an exact determination of Kₐ, though, since sometimes it is sufficient to obtain a qualitative measurement of affinity, e.g., determined using a method such as ELISA or FACS analysis, is proportional to Kₐ, and thus can be used for comparisons, such as determining whether a higher affinity is, e.g., 2, 5, 10, 20, or 50 fold higher than a reference. Binding affinity is typically evaluated in 0.01 M HEPES pH 7.4, 0.15 M NaCl, 3 mM EDTA and 0.005% (v/v) surfactant P20.

An “isolated composition” refers to a composition that is removed from at least 90% of at least one component of a natural sample from which the isolated composition can be obtained. Compositions produced artificially or naturally can be “compositions of at least” a certain degree of purity if the species or population of species of interests is at least 5, 10, 25, 50, 75, 80, 90, 95, 98, or 99% pure on a weight-weight basis.

An “epitope” refers to the site on a target compound that is bound by a ligand, e.g., an antigen-binding protein (e.g., a Fab or antibody). In the case where the target compound is a protein, for example, an epitope may refer to the amino acids that are bound by the ligand. Overlapping epitopes include at least one common amino acid residue.

As used herein, the term “substantially identical” (or “substantially homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the same.

Sequences similar or homologous (e.g., at least about 85% sequence identity) to the sequences disclosed herein are also part of this application. In some embodiments, the sequence identity can be about 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., high stringency hybridization conditions), to the complement of the strand. The nucleic acids may be present in whole cells, in a cell lysate, or in a partially purified or substantially pure form.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. High stringency conditions (3) are the preferred conditions and the ones that should be used unless otherwise specified.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation described herein) are a Blossum 62 scoring matrix with a gap penalty of 12, a gap extend penalty of 4, and a frameshift gap penalty of 5.

As used herein, the terms “homologous” and “homology” are synonymous with “similarity” and means that a sequence of interest differs from a reference sequence by the presence of one or more amino acid substitutions (although modest amino acid insertions or deletions) may also be present. Presently preferred means of calculating degrees of homology or similarity to a reference sequence are through the use of BLAST algorithms (available from the National Center of Biotechnology Information (NCBI), National Institutes of Health, Bethesda Md.), in each case, using the algorithm default or recommended parameters for determining significance of calculated sequence relatedness. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The terms “polypeptide” or “peptide” (which may be used interchangeably) refer to a polymer of three or more amino acids linked by a peptide bond, e.g., between 3 and 30, 12 and 60, or 30 and 300, or over 300 amino acids in length. The polypeptide may include one or more unnatural amino acids. Typically, the polypeptide includes only natural amino acids. A “protein” can include one or more polypeptide chains. Accordingly, the term “protein” encompasses polypeptides. A protein or polypeptide can also include one or more modifications, e.g., a glycosylation, amidation, phosphorylation, and so forth. The term “small peptide” can be used to describe a polypeptide that is between 3 and 30 amino acids in length, e.g., between 8 and 24 amino acids in length.

Statistical significance can be determined by any art known method. Exemplary statistical tests include: the Students T-test, Mann Whitney U non-parametric test, and Wilcoxon non-parametric statistical test. Some statistically significant relationships have a P value of less than 0.05, or 0.02. Particular ligands may show a difference, e.g., in specificity or binding, that are statistically significant (e.g., P value <0.05 or 0.02).

“Angiogenesis-dependent cancers and tumors” are cancers and tumors that require, for their growth (expansion in volume and/or mass), an increase in the number and density of the blood vessels supplying them with blood.

“Regression” refers to the reduction of tumor mass and size, e.g., a reduction of at least 2, 5, 10, or 25%.

### Tie1 Ectodomain-Binding Agents

Tie1 ectodomain-binding agents useful in accordance with the invention bind to an epitope of the Tie1 (e.g., human Tie1 ectodomain). In some embodiments, the Tie1 ectodomain-binding agent increases Tie complex formation. In some embodiments the Tie1 ectodomain-binding agent increases tyrosine phosphorylation of Tie1. In some embodiments, the Tie1 ectodomain-binding agent induces down modulation of Tie1 from the surface of the cell.

Exemplary Tie1 ectodomain binding proteins have been previously disclosed (see, e.g., U.S. Pat. No. 5,955,291 and U.S. Patent Publications Nos. 2005/0136053, 2006/0024297, and 2006/0057138, especially FIGS. 7-39 and Examples 28-30 of U.S. 2006/0057138), and include E3, E3b, M0044-A06; M0044-A11; M0044-B04; M0044-B05; M0044-B08; M0044-B09; M0044-B10; M0044-B12; M0044-C07; M0044-D01; M0044-E03; M0044-F03; M0044-F06; M0044-F09; M0044-G06; M0044-G07; M0044-G11; M0044-H03; M0044-H05; M0044-H07; M0044-H09; M0045-A02; M0045-A04; M0045-B01; M0045-B03; M0045-B11; M0045-C02; M0045-C11; M0045-C12; M0045-D01; M0045-D07; M0045-G01; M0045-G10; M0046-A11; M0046-B06; M0046-B10; M0046-G12; M0046-H03; M0046-H10; M0046-H11; M0047-B03; M0047-D01; M0047-D03; M0047-E10; M0047-G09; M0053-A02; M0053-A03; M0053-A05; M0053-A09; M0053-B09; M0053-B1; M0053-D03; M0053-D06; M0053-D12; M0053-E03; M0053-E04; M0053-E08; M0053-F04; M0053-F05; M0053-F06; M0053-F08; M0053-G04; M0053-G05; M0054-A08; M0054-B06; M0054-B08; M0054-C03; M0054-C07; M0054-E04; M0054-G01; M0054-G05; M0054-H10; M0055-A09; M0055-B11; M0055-B12; M0055-C05; M0055-C07; M0055-D03; M0055-D06; M0055-D12; M0055-E04; M0055-E06; M0055-E10; M0055-E12; M0055-F10; M0055-G02; M0055-G03; M0055-H04; M0056-A01; M0056-A06; M0056-B08; M0056-B09; M0056-C03; M0056-C04; M0056-E08; M0056-F01; M0056-F02; M0056-F10; M0056-F11; M0056-G03; M0056-G04; M0056-G08; M0056-G12; M0056-H04; M0056-H12; M0057-B05; M0057-H07; M0058-A09; M0058-D04; M0058-E09; M0058-F03; M0058-G03; M0058-H01; M0059-A02; M0059-A06; M0060-B02; M0060-H01; M0061-A03; M0061-C05; M0061-C06; M0061-F07; M0061-G12; M0061-H09; M0062-A12; M0062-B05; M0062-B07; M0062-C08; M0062-D04; M0062-E02; M0062-E03; M0062-E11; M0062-F10; M0062-G06; and M0062-H01. Antibody E3 and variants thereof (e.g., DX-2220, DX-2240) and M0044-B08 induce Tie complex formation, Tie1 tyrosine phosphorylation, and down modulation of Tie1 from the cell surface.

Additional or alternate Tie1 ectodomain binding proteins may be isolated using techniques known in the art, including monoclonal antibody production from hybridomas made from B cells isolated from immunized animals (e.g., mice) or selection of display libraries. Display libraries useful for identifying Tie1 ectodomain-binding agents may display peptides (e.g., structured peptides, such as peptides constrained by a disulphide bond; see, e.g., U.S. Patent Publication No. 2006/0084113), or antibodies (e.g., Fabs; see, e.g., Hoet et al., 2005, Nat. Biotech. 23(3):344-48). Tie1 ectodomain (or a portion thereof, such as an EGF domain, a fibronectin repeat, or an Ig-superfamily domain (e.g., a Ig-like C2-type 2 domain)) may be used to identify display library members which bind to the Tie1 ectodomain. For example, Tie1 ectodomain may be recombinantly expressed, attached to a support, then mixed with the display library (e.g., a phage library displaying antibodies). Those members of the library which bind to the Tie1 ectodomain target are then isolated and further characterized. Such techniques are known in the art and are described in U.S. Patent Publication No. 2006/0057138.

Activity of additional/alternate Tie1 ectodomain-binding agents may be assayed using a variety of assays, including the Tie1/EpoR chimeric BaF3 cell assay described in Example 2 of U.S. 2006/0057138. Additional assays include tubulogenesis assays (e.g., Jones M K et al., 1999, Nature Medicine 5:1418-1423), measurements of Tie1 ectodomain-binding agent-induced tyrosine phosphorylation of Tie1 (e.g., phosphorylation of the tyrosine in the motif YVN at about amino acid 1117), as well as in vivo models (e.g., tumor xenograft or orthotopic tumor grafting).

Tie1 ectodomain binding antibodies may be modified in order to make the variable regions of the antibody more similar to one or more germline sequences. For example, an antibody can include one, two, three or more amino acid substitutions, e.g., in a framework or CDR region, to make it more similar to a reference germline sequence. Exemplary germline reference sequences for Vkappa include: O12/O2, O18/O8, A20, A30, L14, L1, L15, L4/18a, L5/L19, L8, L23, L9, L24, L11, L12, O11/O1, A17, A1, A18, A2, A19/A3, A23, A27, A11, L2/L16, L6, L20, L25, B3, B2, A26/A10, and A14. See, e.g., Tomlinson et al. (1995) EMBO J. 14(18):4628-3. A germline reference sequence for the HC variable domain can be based on a sequence that has particular canonical structures, e.g., 1-3 structures in the H1 and H2 hypervariable loops. The canonical structures of hypervariable loops of an immunoglobulin variable domain can be inferred from its sequence, as described in Chothia et al. (1992) J. Mol. Biol. 227:799-817; Tomlinson et al. (1992) J. Mol. Biol. 227:776-798); and Tomlinson et al. (1995) EMBO J. 14(18):4628-38. Exemplary sequences with a 1-3 structure include: DP-1, DP-8, DP-12, DP-2, DP-25, DP-15, DP-7, DP-4, DP-31, DP-32, DP-33, DP-35, DP-40, 7-2, hv3005, hv3005f3, DP-46, DP-47, DP-58, DP-49, DP-50, DP-51, DP-53, and DP-54.

In some embodiments, the Tie1 ectodomain-binding agent is an aptamer. The term nucleic acid “aptamer,” as used herein, refers to a nucleic acid molecule which has a conformation that includes an internal non-duplex nucleic acid structure of at least 5 nucleotides. An aptamer can be a single-stranded nucleic acid molecule which has regions of self-complementarity.

Aptamers can be screened in vitro since a selected aptamer can be recovered by standard nucleic acid amplification procedures. The method can be enhanced, e.g., in later rounds of selection, by splitting selected aptamers into pools and modifying each aptamer in the pool with a detectable label such as a fluorophore. Pools having aptamers that functionally alter the properties of the label can be identified. Such pools can be repeatedly split and reanalyzed to identify the individual aptamers with the desired properties (see, e.g., Jhaveri et al. Nature Biotechnol. 18:1293).

In addition, aptamers can be screened for activity in vivo. For example, shuffled nucleic acids can be cloned into an expression vector that is introduced into cells. RNA aptamers resulting from the expressed shuffled nucleic acids can be screened for a biological activity. Cells having the activity can be isolated and the expression vector for the selected RNA aptamer recovered.

An important feature of therapeutic oligomers (e.g., aptamers) is the design of the backbone of the administered oligomer. In some embodiments, the backbone contains internucleoside linkages that are stable in vivo and is structured such that the oligomer is resistant to endogenous nucleases, such as nucleases that attack the phosphodiester linkage. At the same time, the oligomer retains its ability to hybridize to the target DNA or RNA (Agarwal, K. L. et al. (1979) Nucleic Acids Res. 6:3009; Agarwal, S. et al. (1988) Proc. Natl. Acad. Sci. USA 85:7079). Modified oligonucleotides can be constructed using alternate internucleoside linkages. Several of these exemplary linkages are described in Uhlmann, E. and Peyman, A. (1990) Chemical Reviews 90:543-584. Among these are methylphosphonates (wherein one of the phosphorus-linked oxygens has been replaced by methyl); phosphorothioates (wherein sulphur replaces one of these oxygens) and various amidates (wherein NH₂ or an organic amine derivative, such as morpholidates or piperazidates, replace an oxygen). These substitutions confer enhanced stability. WO 91/15500 teaches various oligonucleotide analogs in which one or more of the internucleotide linkages are replaced by a sulfur based linkage, typically sulfamate diesters, which are isosteric and isoelectric with the phosphodiester. WO 89/12060 similarly discloses linkages containing sulfides, sulfoxides, and sulfones. WO 86/05518 suggests a variant of stereoregular polymeric 3′,5′linkages. U.S. Pat. No. 5,079,151 discloses a msDNA molecule of branched RNA linked to a single strand DNA via a 2′,5′ phosphodiester linkage. U.S. Pat. No. 5,264,562 describes modified linkages of the formula —Y′CX′₂Y′— wherein Y′ is independently O or S and wherein each X′ is a stabilizing substituent and independently chosen. Morpholino-type internucleotide linkages are described in U.S. Pat. No. 5,034,506 and in some cases give rise to an increased affinity of the oligomer for complementary target sequences. U.S. Pat. Nos. 5,264,562 5,596,086 disclose modified oligonucleotides having modified nucleoside linkages which are capable of strong hybridization to target RNA and DNA.

### VDAs

VDAs useful in the instant invention disrupt existing vasculature, particularly the abnormal vasculature associated with angiogenesis-related disorders (e.g., neoplastic disorders). Exemplary VDAs include combretastatins and combretastatin-related compounds, colchicine and colchicine-related compounds, flavone-related compounds, dolastatin 10 derivatives, other microtubule disrupting agents and agents which target abnormal vasculature.

Combretastatin-related compounds include combretastatin A-4 disodium phosphate (CA4P), combretastatin analogs such as (Z)-N-[2-methoxy-5-[2-(3,4,5-trimethoxyphenyl)vinyl]phenyl]-L-serinamide hydrochloride (AVE8062, Sanofi-Aventis), and combretastatin A-1 prodrugs such as CA-1-P (also known as OXI4503, Oxigene).

Colchicine-related compounds include N-acetylcolchinol (5S)-5-(acetylamino)-9,10,11-trimethoxy-6,7-dihydro-5H-dibenzo[a,c]cyclohepten-3-yl dihydrogenphosphate (ZD6126, AstraZeneca).

Flavone-related compounds include flavone acetic acid (FAA) and tricyclic analogues of FAA, such as 5,6-dimethylxanthenone-4-acetic acid (DMXAA, also known as AS1404, Antisoma).

Dolastatin 10-related compounds include soblidotin (N²—(N,N-dimethyl-L-valyl)-N-[(1S,2R)-2-methoxy-4-[(2S)-2-[(1R,2R)-1-methoxy-2-methyl-3-oxo-3-[(2-phenylethyl)]amino]propyl]-1-pyrrolidinyl]-1-[(S)-1-methylpropyl]-4-oxobutyl]-N-methyl-L-valinamide, also known as TZT1027).

Additional VDAs include BNC105 (Bionomics Ltd.) and microtubule disrupting agents such as MPC-6827 (Myriad Genetics), CYT997 (Cytopia Ltd.).

### Protein Production

Standard recombinant nucleic acid methods can be used to express Tie1 ectodomain binding proteins and VDAs which are proteins. See, for example, the techniques described in Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989). Generally, a nucleic acid sequence encoding the binding protein is cloned into a nucleic acid expression vector. If the protein includes multiple polypeptide chains, each chain can be cloned into an expression vector, e.g., the same or different vectors, that are expressed in the same or different cells. Methods for producing antibodies are also provided below.

Some antibodies, e.g., Fabs, can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be shuffled into a bacterial cell that cannot suppress a stop codon. In this case, the Fab is not fused to the gene III protein and is secreted into the media.

Antibodies can also be produced in eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one embodiment, antibodies are produced in mammalian cells. Preferred mammalian host cells for expressing the clone antibodies or antigen-binding fragments thereof include Chinese Hamster Ovary (CHO cells) (including dhfr− CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells, SP2 cells, COS cells, HEK 293T cells, and a cell from a transgenic animal, e.g., a transgenic mammal. For example, the cell is a mammary epithelial cell.

In addition to the nucleic acid sequence encoding the immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically, the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection). Another exemplary expression system is the glutamine synthase (GS) vector system available from Lonza Group Ltd. CH (see, e.g., Clark et al. (2004) BioProcess International 2(4):48-52; Barnes et al. (2002) Biotech Bioeng. 81(6):631-639).

In an exemplary system for recombinant expression of an antibody, or antigen-binding portion thereof, a recombinant expression vector encoding both the antibody heavy chain and the antibody light chain is introduced into dhfr− CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G.

The codon usage can be adapted to the codon bias of the host cell, e.g., for CHO cells it can be adapted for the codon bias for Cricetulus griseus genes. In addition, regions of very high (>80%) or very low (<30%) GC content can be avoided where possible. During the optimization process the following cis-acting sequence motifs were avoided: internal TATA-boxes; chi-sites and ribosomal entry sites; AT-rich or GC-rich sequence stretches; ARE, INS, CRS sequence elements; repeat sequences and RNA secondary structures; and (cryptic) splice donor and acceptor sites, branch points. Two STOP codons can be used to ensure efficient termination. The codon optimization of the sequence can be evaluated according to Sharp, P. M., Li, W. H., Nucleic Acids Res. 15 (3), 1987). The standard codon adaptation index (CAI) can be used. Rare codons include those with a quality class between 0-40.

Codon-altered (e.g., codon-optimized) sequences can be used to produce an antibody. An exemplary method includes providing a mammalian cell that includes an antibody-coding nucleic acid and expressing the nucleic acid in the cell, e.g., maintaining the cell under conditions in which the protein is expressed. The antibody-coding nucleic acid can be providing in a mammalian expression vector, e.g., a vector that is introduced into the cell. The cell can be a non-human mammalian cell, e.g., a CHO cell.

For antibodies that include an Fc domain, the antibody production system preferably synthesizes antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof (1992) Adv. Immunol. 51:1-84; Jefferis et al. (1998) Immunol. Rev. 163:59-76). In a preferred embodiment, the Fc domain is produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

It is also possible to produce antibodies that bind to Tie1 ectodomain by immunization, e.g., using an animal, e.g., with natural, human, or partially human immunoglobulin loci. Such an antibody can be of any allotype, e.g., a,z allotype, f allotype, or non-A allotype. Non-human antibodies can also be modified to include substitutions for human immunoglobulin sequences, e.g., consensus human amino acid residues at particular positions, e.g., at one or more of the following positions (preferably at least five, ten, twelve, or all): (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213.

### Pharmaceutical Compositions

The Tie1 ectodomain-binding agent and VDA are typically administered in the methods of the invention as pharmaceutical compositions. A “pharmaceutical composition” of an agent is the agent formulated with a pharmaceutically acceptable carrier. Pharmaceutical compositions encompass labeled binding proteins (e.g., for in vivo imaging) as well as therapeutic compositions.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the binding protein, may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. For Tie1 ectodomain-binding agents and VDAs that are proteins, the typical preferred formulations are in the form of injectable or infusible solutions, such as compositions similar to those used for administration of humans with antibodies. For such proteinaceous agents, the preferred mode of administration is typically parenteral (e.g., intravenous, subcutaneous, intraperitoneal, intramuscular). In some embodiments, the proteinaceous Tie1 ectodomain-binding agent and/or VDA is administered by intravenous infusion, e.g., at a rate of less than 30, 20, 10, 5, or 1 mg/min to reach a dose of about 1 to 100 mg/m² or 7 to 25 mg/m², or by injection. In other embodiments, the proteinaceous Tie1 ectodomain-binding agent and/or VDA is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The Tie1 ectodomain-binding agent and/or VDA can be formulated as a solution, microemulsion, dispersion, liposome, or other ordered structure suitable to high drug concentration. Sterile injectable solutions can be prepared by incorporating the agent in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In certain embodiments, the binding protein may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound described herein by other than parenteral administration, it may be necessary to coat the compound with, or co-administer the compound with, a material to prevent its inactivation.

Pharmaceutical compositions can be administered with medical devices known in the art. For example, in a preferred embodiment, a pharmaceutical can be administered with a needleless hypodermic injection device, such as the devices disclosed in U.S. Pat. No. 5,399,163, 5,383,851, 5,312,335, 5,064,413, 4,941,880, 4,790,824, or 4,596,556. Examples of implants and modules include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

In certain embodiments, a binding protein described herein can be formulated to ensure proper distribution in vivo. For example, the blood-brain barrier (BBB) excludes many highly hydrophilic compounds. To ensure that the therapeutic protein crosses the BBB (if desired), it can be formulated, for example, in liposomes. For methods of manufacturing liposomes, see, e.g., U.S. Pat. Nos. 4,522,811; 5,374,548; and 5,399,331. The liposomes may include one or more moieties which are selectively transported into specific cells or organs, thus enhance targeted drug delivery (see, e.g., V. V. Ranade (1989) J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms can be dictated by and directly dependent on (a) the unique characteristics of the active compound and the particular therapeutic effect to be achieved, and (b) the limitations inherent in the art of compounding such an active compound for the treatment of sensitivity in individuals.

The pharmaceutical compositions may be prepared using a “therapeutically effective amount” or a “prophylactically effective amount” of a target-binding protein described herein. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the binding protein to elicit a desired response in the individual. A therapeutically effective amount is also one in which any toxic or detrimental effects of the composition are outweighed by the therapeutically beneficial effects. A “therapeutically effective dosage” preferably inhibits a measurable parameter, e.g., inflammation or tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

As used herein, “a prophylactically effective amount” refers to an amount effective, at dosages and for periods of time necessary, in preventing or delaying the occurrence of the onset or recurrence of a disorder, e.g., an angiogenesis-related disorder described herein.

Also within the scope of the invention are kits including (a) a Tie1 ectodomain-binding agent, (b) a VDA, and (c) instructions for use in accordance with the methods disclosed herein. The instructions for therapeutic applications include suggested dosages and/or modes of administration in a patient with (a) a cancer or neoplastic disorder, (b) an inflammatory disorder (e.g., rheumatoid arthritis), or an ocular disorder. The kit can further contain at least one additional reagent, such as an additional therapeutic agent, (e.g., a cytotoxic chemotherapy agent) formulated as appropriate, in one or more separate pharmaceutical preparations.

### Stabilization and Retention

In some embodiments, the Tie1 ectodomain-binding agent or VDA is physically associated with a moiety that improves its stabilization and/or retention in circulation, e.g., in blood, serum, lymph, or other tissues.

For example, the Tie1 ectodomain-binding agent or VDA can be associated with a polymer, e.g., a substantially non-antigenic polymers, such as polyalkylene oxides or polyethylene oxides. Suitable polymers will vary substantially by weight. Exemplary polymers include polymers having molecular number average weights ranging from about 200 to about 35,000, from about 1,000 to about 15,000, and 2,000 to about 12,500, but can range higher, (e.g., up to about 500,000 D), and in some embodiments is at least about 20,000 D, or at least about 30,000 D, or at least about 40,000 D. The molecular weight chosen can depend upon the effective size of the conjugate to be achieved, the nature (e.g. structure, such as linear or branched) of the polymer, and the degree of derivatization.

Polymers useful for modification of the Tie1 ectodomain-binding agent and/or VDA include water soluble polymers for, e.g., hydrophilic polyvinyl polymers, e.g. polyvinylalcohol and polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g., polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparan.

Other compounds can also be attached to the same polymer, e.g., a cytotoxin, a label, or another targeting agent, e.g., another target-binding agent or an unrelated agent. Mono-activated, alkoxy-terminated polyalkylene oxides (PAO's), e.g., monomethoxy-terminated polyethylene glycols (mPEG's); C₁₋₄ alkyl-terminated polymers; and bis-activated polyethylene oxides (glycols) can be used for crosslinking. See, e.g., U.S. Pat. No. 5,951,974.

In its most common form poly(ethylene glycol), PEG, is a linear or branched polyether terminated with hydroxyl groups and having the general structure:

HO—(CH₂CH₂O)ₙ—CH₂CH₂—OH

PEG can be synthesized by anionic ring opening polymerization of ethylene oxide initiated by nucleophilic attack of a hydroxide ion on the epoxide ring. Particularly useful for polypeptide modification is monomethoxy PEG, mPEG, having the general structure:

CH₃O—(CH₂CH₂O)ₙ—CH₂CH₂—OH

For further description, see, e.g., Roberts et al. (2002) Advanced Drug Delivery Reviews 54:459-476.

The covalent crosslink can be used to attach a target-binding agent (e.g., a protein) to a polymer, for example, crosslinking to the N-terminal amino group and epsilon amino groups found on lysine residues, as well as other amino, imino, carboxyl, sulfhydryl, hydroxyl or other hydrophilic groups. The polymer may be covalently bonded directly to the target-binding protein without the use of a multifunctional (ordinarily bifunctional) crosslinking agent. Covalent binding to amino groups is accomplished by known chemistries based upon cyanuric chloride, carbonyl diimidazole, aldehyde reactive groups (PEG alkoxide plus diethyl acetal of bromoacetaldehyde; PEG plus DMSO and acetic anhydride, or PEG chloride plus the phenoxide of 4-hydroxybenzaldehyde, activated succinimidyl esters, activated dithiocarbonate PEG, 2,4,5-trichlorophenylcloroformate or P-nitrophenylcloroformate activated PEG). Carboxyl groups can be derivatized by coupling PEG-amine using carbodiimide. Sulfhydryl groups can be derivatized by coupling to maleimido-substituted PEG (e.g. alkoxy-PEG amine plus sulfosuccinimidyl 4-(N-maleimidomethyl)cyclohexane-1-carboxylate) (WO 97/10847) or PEG-maleimide commercially available from Shearwater Polymers, Inc., Huntsville, Ala.). Alternatively, free amino groups on the binding protein (e.g. epsilon amino groups on lysine residues) can be thiolated with 2-imino-thiolane (Traut's reagent) and then coupled to maleimide-containing derivatives of PEG, e.g., as described in Pedley et al., Br. J. Cancer, 70: 1126-1130 (1994).

Functionalized PEG polymers that can be attached to a Tie1 ectodomain-binding agent or a VDA are available, e.g., from Shearwater Polymers, Inc. (Huntsville, Ala.). Such commercially available PEG derivatives include, e.g., amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives may vary depending on the Tie1 ectodomain-binding agent or VDA involved, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc. Specific instructions for the use of any particular derivative are available from the manufacturer.

The conjugates of a Tie1 ectodomain-binding agent or a VDA and a polymer can be separated from the unreacted starting materials, e.g., by gel filtration or ion exchange chromatography, e.g., HPLC. Heterologous species of the conjugates are purified from one another in the same fashion. Resolution of different species (e.g., containing one or two PEG residues) is also possible, e.g., due to the difference in the ionic properties of unreacted amino acids. See, e.g., WO 96/34015.

A target binding protein can also be physically associated with a protein that provides a stabilizing or retention function, e.g., an albumin, e.g., human serum albumin. US 2004/0171794 describes exemplary methods for physically associating a protein with serum albumin. For exemplary, human albumin sequences or fragments thereof, see EP 201 239, EP 322 094 WO 97/24445, WO95/23857 especially the mature form of human albumin as shown in SEQ ID NO:18 of US 2004/0171794 and WO 01/79480 or albumin from other vertebrates or fragments thereof, or analogs or variants of these molecules or fragments thereof. Other exemplary human serum albumin proteins can include one or both of the following sets of point mutations Leu-407 to Ala, Leu-408 to Val, Val-409 to Ala, and Arg-410 to Ala; or Arg-410 to Ala, Lys-413 to Gln, and Lys-414 to Gln (see, e.g., International Publication No. WO 95/23857, with reference to SEQ ID NO:18 of US 2004/0171794).

### Methods of Use

The invention provides methods for treating at least one symptom of an angiogenesis-related disorder by administering a Tie1 ectodomain-binding agent and a VDA.

The Tie1 ectodomain-binding agent may be administered prior to initiation of administration of the VDA. The period of administration of the Tie1 ectodomain-binding agent may be less than one day (e.g., less than one hour, or about 1, 2, 4, 6, 8, 12, 18 or 24 hours) or range from about 1 day up to about 35 days (e.g., 5, 7, 10, 14, 20, 21, 28, 30, or 35 days, and days or ranges in between) prior to the first administration of the VDA. The period can be calculated based, e.g., upon the first administration of the Tie1 ectodomain-binding agent for a given treatment cycle. The period of Tie1 ectodomain-binding protein administration may be followed by hiatus period during which neither the Tie1 ectodomain-binding protein nor the VDA are administered. The hiatus period may be may be less than one day (e.g., less than one hour, or about 1, 2, 3, 4, 6, 8, 12, 18, or 24 hours) or range from 1 day up to 35 days (e.g., 1, 2, 3, 4, 5, 6, 7, 8, 10, 12, 14, 20, 21, 28, 30, or 35 days, or any day or range in between) prior to the first administration of the VDA.

Conversely, the Tie1 ectodomain-binding agent may be administered following first administration of the VDA. The period between first administration of the VDA and administration of the Tie1 ectodomain-binding agent may be less than one day (e.g., less than one hour, or about 1, 2, 3, 4, 6, 8, 12, 18, or 24 hours) or range from 1 day up to 35 days (e.g., 1, 2, 3, 4, 5, 6, 7, 8, 10, 12, 14, 20, 21, 28, 30, or 35 days, or any day or range in between). The period of VDA administration may be followed by hiatus period during which neither the VDA nor the Tie1 ectodomain-binding agent are administered. The hiatus period may be may be less than one day (e.g., less than one hour, or about 1, 2, 3, 4, 6, 8, 12, 18, or 24 hours) or range from 1 day up to 35 days (e.g., 1, 2, 3, 4, 5, 6, 7, 8, 10, 12, 14, 20, 21, 28, 30, or 35 days, or any day or range in between) prior to the first administration of the VDA.

The combination therapy disclosed herein may be administered in a series (two or more) of cycles. For example, in configurations in which the Tie1 ectodomain-binding agent is administered for a period prior to the initiation of administration of the VDA and the Tie1 ectodomain-binding agent is discontinued upon, or following, the initiation of VDA administration, the Tie1 ectodomain-binding agent may be reinitiated following completion of administration of the VDA.

The Tie ectodomain-binding agent and the VDA may be administered on an alternating schedule. For example, the Tie1 ectodomain-binding agent is administered for a period, followed by administration of a VDA, followed by further administration of the Tie1 ectodomain-binding agent, followed by another administration of the VDA, etc. The converse schedule may also be used (VDA, then Tie1 ectodomain-binding agent, then VDA, then Tie1 ectodomain-binding agent, etc.).

Additionally, the Tie1 ectodomain-binding agent and the VDA may be given simultaneously (e.g., administration of the two agents is initiated within about 10 minutes).

In configurations in which the Tie1 ectodomain-binding agent and the VDA are administered sequentially, the first administered agent may be continued or discontinued following initiation of administration of the second agent (e.g., when administration of the VDA is initiated prior to initiation of administration of the Tie1 ectodomain-binding agent, the VDA treatment may be continued or discontinued).

In some embodiments, the subject is in need of reduced angiogenesis, or identified as such. For example, the subject has a neoplastic disorder, e.g., a metastatic cancer. For example, the subject has an angiogenesis-dependent cancer or tumor. The tumor can be a solid tumor, e.g., a tumor at least 1, 2, 3, 5, 8 or 10 mm in diameter. In one embodiment, the solid tumor has a hypoxic core. The method can include, prior to administering the antagonist, evaluating the subject and detecting a solid tumor in the subject.

In some embodiments, the Tie1 ectodomain-binding agent and the VDA are each administered in an amount effective to individually reduce angiogenesis in the subject or otherwise treat a disorder in a subject (e.g., ameliorate a symptom of the disorder). In other embodiments, the Tie1 ectodomain-binding agent and the VDA are each administered in an amount that is less than an amount effective to individually reduce angiogenesis in the subject or otherwise treat or prevent a disorder in a subject. In other embodiments, the VDA is administered in an amount that is less than an amount effective to individually reduce angiogenesis in the subject or otherwise treat or prevent a disorder in a subject. In other embodiments, the Tie1 ectodomain-binding agent is administered in an amount that is less than an amount effective to individually reduce angiogenesis in the subject or otherwise treat or prevent a disorder in a subject. In some embodiments, the Tie1 ectodomain-binding agent and the VDA are administered in synergistically effective amounts (e.g., amounts which, when compared to either compound administered alone, result in a synergistic effect).

Angiogenesis-related disorders include, but are not limited to, neoplastic disease (e.g., solid tumors, tumor metastases, and benign tumors, particularly neoplastic disease requiring a blood supply or angiogenesis); inflammatory disorders (e.g., rheumatoid arthritis, lupus, restenosis, psoriasis, graft v. host response, or multiple sclerosis); ocular angiogenic diseases, for example, retinal disorders (e.g., a proliferative retinopathy, such as diabetic retinopathy, ischemic retinopathy, or retinopathy of prematurity); choroidal neovascularization; lens neovascularization; corneal neovascularization; iridial neovascularization; or conjunctival neovascularization, macular degeneration (e.g., wet and/or dry forms of age-related macular degeneration), corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation.

Benign tumors include, but are not limited to hemangiomas, acoustic neuromas, neurofibromas, trachomas, and pyogenic granulomas. Solid tumors include, but are not limited to malignancies, e.g., sarcomas, adenocarcinomas, and carcinomas, of the various organ systems, such as those affecting lung, breast, gastrointestinal (e.g., colon), and genitourinary tract (e.g., renal, urothelial cells), pharynx, as well as adenocarcinomas which include malignancies such as most colon cancers, rectal cancer, renal-cell carcinoma, liver cancer, non-small cell carcinoma of the lung, cancer of the small intestine, cancer of the esophagus, and pancreatic cancer. Still further examples of solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In some embodiments, administration of a Tie1 ectodomain-binding agent and a VDA, as described herein, can ameliorate a symptom of a disorder, e.g., an angiogenesis-related disorder (e.g., as described herein). When the angiogenesis-related disorder is a neoplastic disorder, amelioration of a symptom of the disorder is elimination, reduction, stabilization, as determined by clinical measurements (e.g., magnetic resonance imaging (MRI), computed tomography (CT), diagnostic radiometry (e.g., bone scan), and the like) in the amount of the neoplastic disorder (e.g., tumor size), or reduction in the growth rate or number of tumors (e.g., metastases). Other parameters that can be affected include activities of daily living, pain (e.g., patient reported pain using a visual or numerical scale).

In some embodiments, the angiogenesis-related disorder is an inflammatory disorder, e.g., rheumatoid arthritis, psoriasis, rheumatoid or rheumatic inflammatory disease, or other chronic inflammatory disorders, such as chronic asthma, arterial or post-transplantational atherosclerosis, and endometriosis. Other angiogenesis-related disorders that can be treated include those that have deregulated or undesired angiogenesis, such as ocular neovascularization, e.g., retinopathies (including diabetic retinopathy and age-related macular degeneration) hemangioblastoma, hemangioma, and arteriosclerosis.

Psoriasis is a chronic skin disease, characterized by scaling and inflammation. When psoriasis develops, typically patches of skin thicken, redden, and become covered with silvery scales, referred to as plaques. Psoriasis most often occurs on the elbows, knees, scalp, lower back, face, palms, and soles of the feet. The disease also may affect the fingernails, toenails, and the soft tissues inside the mouth and genitalia. About 10 percent of people with psoriasis have joint inflammation that produces symptoms of arthritis. Patients can be evaluated using a static Physician Global Assessment (sPGA), and receive a category score ranging from six categories between clear and very severe. The score is based on plaque, scaling, and erythema. The therapeutic methods herein can be used to achieve an improvement for at least one of these indicia.

Rheumatoid arthritis (“RA”) is a chronic inflammatory disease that causes pain, swelling, stiffness, and loss of function, primarily the joints. RA frequently begins in the synovium, the membrane that surrounds a joint creating a protective sac. In many individuals suffering from RA, leukocytes infiltrate from the circulation into the synovium causing continuous abnormal inflammation (e.g., synovitis). Consequently, the synovium becomes inflamed, causing warmth, redness, swelling, and pain. The collagen in the cartilage is gradually destroyed, narrowing the joint space and eventually damaging bone. The inflammation causes erosive bone damage in the affected area. During this process, the cells of the synovium grow and divide abnormally, making the normally thin synovium thick and resulting in a joint that is swollen and puffy to the touch. RA can be assessed by a variety of clinical measures. Some exemplary indicia include the total Sharp score (TSS), Sharp erosion score, and the HAQ disability index. The therapeutic methods herein can be used to achieve an improvement for at least one of these indicia.

As used herein, an amount of a Tie1 ectodomain-binding agent or VDA effective to treat (e.g., ameliorate at least one symptom of) a disorder, or a “therapeutically effective amount” refers to an amount of the Tie1 ectodomain-binding agent or VDA which is effective, upon single or multiple-dose administration to a subject, in prolonging curing, alleviating, relieving or improving a subject with a disorder as described herein beyond that expected in the absence of such treatment. In some cases, a therapeutically effective amount can be ascertained by evaluating the ability of the binding agent to reduce tumor size of a xenograft in a nude mouse model relative to an untreated control mouse. As used herein, “inhibiting the growth” of a tumor or other neoplasm refers to slowing, interrupting, arresting or stopping its growth and metastases and does not necessarily indicate a total elimination of the neoplastic growth.

An exemplary, non-limiting range for a therapeutically effective amount of an antibody described herein is 0.1-20 mg/kg, more preferably 1-10 mg/kg. The target-binding antibody can be administered by intravenous infusion at a rate of less than 30, 20, 10, 5, or 1 mg/min to reach a dose of about 1 to 100 mg/m² or about 5 to 30 mg/m². For Tie1 ectodomain-binding agents smaller in molecular weight than an antibody, appropriate amounts can be proportionally less.

The exemplary VDAs disclosed herein are generally considered ‘small molecule’ therapeutics, and thus the dosage ranges will depend on the pharmacological characteristics of the particular VDA, as well as considerations of patient size (e.g., body surface area) and other parameters.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Subjects that can be treated include human and non-human animals. For example, the human can be a human patient having a disorder characterized by abnormal cell proliferation or cell differentiation. The term “non-human animals” includes all vertebrates, e.g., non-mammals (such as chickens, amphibians, reptiles) and mammals, such as non-human primates, sheep, dog, cow, pig, etc.

Methods of administering Tie1 ectodomain-binding agents, VDAs and other agents (e.g., cytotoxic chemotherapy agents) are also described in “Pharmaceutical Compositions”. Suitable dosages of the molecules used will depend on the age and weight of the subject and the particular drug used.

### Combination Therapies

The treatment methods disclosed herein can be used in combination with one or more additional treatment modalities, including, but not limited to: surgery; radiation therapy, and chemotherapy.

With reference to the methods disclosed herein, the term “combination” refers to the use of one or more additional agents or therapies to treat the same patient, wherein the use or action of the agents or therapies overlap in time. The additional agents or therapies can be administered at the same time as the Tie1 ectodomain binding protein and/or VDA are administered, or sequentially in any order. Sequential administrations are administrations that are given at different times. The time between administration of the one agent and another agent can be minutes, hours, days, or weeks.

The additional agent or therapy can also be another anti-cancer agent or therapy. Nonlimiting examples of anti-cancer agents include, e.g., anti-angiogenic agents, anti-microtubule agents, topoisomerase inhibitors, antimetabolites, mitotic inhibitors, alkylating agents, intercalating agents, agents capable of interfering with a signal transduction pathway, agents that promote apoptosis, radiation, and antibodies against other tumor-associated antigens (including naked antibodies, immunotoxins and radioconjugates). Examples of the particular classes of anti-cancer agents are provided in detail as follows: anti-angiogenic agents, e.g., VEGF pathway antagonists (agents that targets or negatively regulate the VEGF signaling pathway) including VEGF inhibitors (e.g., agents that directly inhibit VEGF (e.g., VEGF-A, -B, or -C), such as by binding VEGF (e.g., anti-VEGF antibodies such as bevacizumab (AVASTIN®) or ranibizumab, or other inhibitors such as pegaptanib, ranibizumab, NEOVASTAT®, AE-941, VEGF Trap, and PI-88)), modulators of VEGF expression (e.g., INGN-241, oral tetrathiomolybdate, 2-methoxyestradiol, 2-methoxyestradiol nanocrystal dispersion, bevasiranib sodium, PTC-299, Veglin), inhibitors of a VEGF receptor (e.g., KDR or VEGF receptor III (Flt4), for example anti-KDR antibodies, VEGFR2 antibodies such as CDP-791, IMC-1121B, VEGFR2 blockers such as CT-322), modulators of VEGFR expression (e.g., VEGFR1 expression modulator Sirna-027) or inhibitors of VEGF receptor downstream signaling. In some embodiments, the VEGF antagonist agent is bevacizumab, pegaptanib, ranibizumab, sorafenib, sunitinib, NEOVASTAT®, AE-941, VEGF Trap, pazopanib, vandetanib, vatalanib, cediranib, fenretinide, squalamine, INGN-241, oral tetrathiomolybdate, tetrathiomolybdate, Panzem NCD, 2-methoxyestradiol, AEE-788, AG-013958, bevasiranib sodium, AMG-706, axitinib, BIBF-1120, CDP-791, CP-547632, PI-88, SU-14813, SU-6668, XL-647, XL-999, IMC-1121B, ABT-869, BAY-57-9352, BAY-73-4506, BMS-582664, CEP-7055, CHIR-265, CT-322, CX-3542, E-7080, ENMD-1198, OSI-930, PTC-299, Sirna-027, TKI-258, Veglin, XL-184, or ZK-304709; antitubulin/antimicrotubule agent, e.g., paclitaxel, vincristine, vinblastine, vindesine, vinorelbin, taxotere; topoisomerase I inhibitors, e.g., irinotecan, topotecan, camptothecin, doxorubicin, etoposide, mitoxantrone, daunorubicin, idarubicin, teniposide, amsacrine, epirubicin, merbarone, piroxantrone hydrochloride; antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5-fluorouridine, FUDR, tiazofurin, N-[5-[N-(3,4-dihydro-2-methyl-4-oxoquinazolin-6-ylmethyl)-N-methylamino]-2-thenoyl]-L-glutamic acid; alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol; agents acting via other mechanisms of action, e.g., dihydrolenperone, spiromustine, and desipeptide; biological response modifiers, e.g., to enhance anti-tumor responses, such as interferon; apoptotic agents, such as actinomycin D; and anti-hormones, for example anti-estrogens such as tamoxifen or, for example antiandrogens such as 4′-cyano-3-(4-fluorophenylsulphonyl)-2-hydroxy-2-methyl-3′-(trifluoromethyl) propionanilide.

A combination therapy can include administering an agent that reduces the side effects of other therapies. The agent can be an agent that reduces the side effects of anti-cancer treatments. For example, the agent can be leucovorin (e.g., in combination with 5-fluorouracil).

The following examples are not to be construed as limiting.

### EXAMPLES

### Example 1

### Exemplary Tie1 Ectodomain-Binding Antibody Sequences

The following are exemplary sequences of immunoglobulin light chain and heavy chain variable domains:

806C-M0044-B08
L-Variable (AA):

(SEQ ID NO:1)

QDIQMTQSPSFLSASVGDRVTISCRASQYISIYLNWYQQRPGEAPKLLIN
AASSLQSGDPSRFSGSGSGTDFTLTINSLQPDDFATYYCQQYKSYPLTFG
EGTKVEIK
L-Variable (DNA):

(SEQ ID NO:2)

CAAGACATCCAGATGACCCAGTCTCCATCCTTCCTGTCCGCATCTGTAGG
AGACAGAGTCACCATCTCTTGCCGGGCAAGTCAGTACATCAGCATATATT
TGAATTGGTATCAGCAGAGACCAGGGGAAGCCCCTAAACTCCTGATCAAT
GCTGCATCCAGTTTGCAAAGTGGGGACCCATCAAGGTTCAGTGGCAGTGG
ATCTGGGACAGATTTCACTCTCACCATCAACAGCCTGCAGCCTGATGATT
TTGCAACTTATTACTGCCAACAGTATAAGAGTTACCCCCTCACTTTCGGC
GAGGGGACCAAGGTGGAGATCAAA
H-Variable (AA):

(SEQ ID NO:3)

EVQLLESGGGLVQPGGSLRLSCAASGFTFSAYGMGWVRQAPGKGLEWVSV
ISPSGGQTSYADSVKGRFTISRDNSKNTLYLQMNSLRAEDTALYYCAGGD
RYGPLHYWGQGTLVTVSS
H-Variable (DNA):

(SEQ ID NO:4)

GAAGTTCAATTGTTAGAGTCTGGTGGCGGTCTTGTTCAGCCTGGTGGTTC
TTTACGTCTTTCTTGCGCTGCTTCCGGATTCACTTTCTCTGCTTACGGTA
TGGGTTGGGTTCGCCAAGCTCCTGGTAAAGGTTTGGAGTGGGTTTCTGTT
ATCTCTCCTTCTGGTGGCCAGACTTCTTATGCTGACTCCGTTAAAGGTCG
CTTCACTATCTCTAGAGACAACTCTAAGAATACTCTCTACTTGCAGATGA
ACAGCTTAAGGGCTGAGGACACCGCCTTGTATTACTGTGCGGGAGGGGAC
AGGTATGGACCCTTGCACTACTGGGGCCAGGGAACCCTGGTCACCGTCTC
AAGC

DX-2220

DX-2220 is a full length, IgG1, germlined human anti-Tie1 antibody E3b. The sequence of DX-2220 is as follows:

DX-2220 Light Chain Amino Acid Sequence:

(SEQ ID NO:700)

DIQMTQSPSSLSASVGDRVTITCRASQGIGHYLAWYQQKPGKVPKLLIYT
ASTLQSGVPSRFSGSGSGTDFTLTISSLQPEDVATYYCQQFNSYPHTFGQ
GTRLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
DX-2220 Heavy Chain Amino Acid Sequence:

(SEQ ID NO:701)

EVQLLESGGGLVQPGGSLRLSCAASGFTFSMYGMVWVRQAPGKGLEWVSV
ISPSGGNTGYADSVKGRFTISRDNSKNTLYLQMNSLRAEDTAVYYCARAP
RGYSYGYYYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
An exemplary DX-2220 Light Chain Nucleotide
Sequence:

(SEQ ID NO:702)

ggcgtgcactctgacatccagatgacccagtctccatcctccctgtctgc
atctgtaggagacagagtcaccatcacttgccgggcgagtcagggcattg
gccattatttagcctggtatcagcagaaaccagggaaagttcctaagctc
ctgatctatactgcatccactttgcaatcaggggtcccatctcggttcag
tggcagtggatctgggacagatttcactctcaccatcagcagcctgcagc
ctgaagatgttgcaacttattactgtcaacagtttaatagttaccctcac
accttcggccaagggacacgactggagattaaacgaactgtggctgcacc
atctgtcttcatcttcccgccatctgatgagcagttgaaatctggaactg
cctctgttgtgtgcctgctgaataacttctatcccagagaggccaaagta
cagtggaaggtggataacgccctccaatcgggtaactcccaggagagtgt
cacagagcaggacagcaaggacagcacctacagcctcagcagcaccctga
cgctgagcaaagcagactacgagaaacacaaagtctacgcctgcgaagtc
acccatcagggcctgagctcgcccgtcacaaagagcttcaacaggggaga
gtgttaataa
An exemplary DX-2220 Heavy Chain Nucleotide
Sequence:

(SEQ ID NO:703)

gaagttcaattgttagagtctggtggcggtcttgttcagcctggtggttc
tttacgtctttcttgcgctgcttccggattcactttctctatgtacggta
tggtttgggttcgccaagctcctggtaaaggtttggagtgggtttctgtt
atctctccttctggtggcaatactggttatgctgactccgttaaaggtcg
cttcactatctctagagacaactctaagaatactctctacttgcagatga
acagcttaagggctgaggacactgcagtctactattgtgcgagagcccca
cgtggatacagctatggttactactactggggccagggaaccctggtcac
cgtctcaagcgcctccaccaagggcccatcggtcttcccgctagcaccct
cctccaagagcacctctgggggcacagcggccctgggctgcctggtcaag
gactacttccccgaaccggtgacggtgtcgtggaactcaggcgccctgac
cagcggcgtccacaccttcccggctgtcctacagtcctccggactctact
ccctcagcagcgtagtgaccgtgccctccagcagcttgggcacccagacc
tacatctgcaacgtgaatcacaagcccagcaacaccaaggtggacaagaa
agttgagcccaaatcttgtgacaaaactcacacatgcccaccgtgcccag
cacctgaactcctggggggaccgtcagtcttcctcttccccccaaaaccc
aaggacaccctcatgatctcccggacccctgaggtcacatgcgtggtggt
ggacgtgagccacgaagaccctgaggtcaagttcaactggtacgtggacg
gcgtggaggtgcataatgccaagacaaagccgcgggaggagcagtacaac
agcacgtaccgtgtggtcagcgtcctcaccgtcctgcaccaggactggct
gaatggcaaggagtacaagtgcaaggtctccaacaaagccctcccagccc
ccatcgagaaaaccatctccaaagccaaagggcagccccgagaaccacag
gtgtacaccctgcccccatcccgggatgagctgaccaagaaccaggtcag
cctgacctgcctggtcaaaggcttctatcccagcgacatcgccgtggagt
gggagagcaatgggcagccggagaacaactacaagaccacgcctcccgtg
ctggactccgacggctccttcttcctctacagcaagctcaccgtggacaa
gagcaggtggcagcaggggaacgtcttctcatgctccgtgatgcatgagg
ctctgcacaaccactacacgcagaagagcctctccctgtctccgggtaaa
tga

### Example 2

### Sequence of DX-2240

### Germlined F Allotyped E3 Antibody

DX-2240 (Light, heavy-variable, constant).
Variable region:

(SEQ ID NO:714)

DIQMTQSPSSLSASVGDRVTITCRASQGIGHYLAWYQQKPGKVPKLLIYT
ASTLQSGVPSRFSGSGSGTDFTLTISSLQPEDVATYYCQQFNSYPHTFGQ
GTRLEIK
Light constant:
SEQ ID NO:724 light chain (variable + constant)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
DX-2240 Heavy variable:

(SEQ ID NO:725)

EVQLLESGGGLVQPGGSLRLSCAASGFTFSMYGMVWVRQAPGKGLEWVSV
ISPSGGNTGYADSVKGRFTISRDNSKNTLYLQMNSLRAEDTAVYYCARAP
RGYSYGYYYWGQGTLVTVSS
Heavy constant (CH1, Hinge, CH2, CH3):
SEQ ID NO:723 heavy chain (variable + constant)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

The light chain can optionally further include the following signal sequence: Light signal sequence: MGWSCIILFLVATATGVHS (SEQ ID NO:729). The heavy chain can optionally further include the following signal sequence MGWSCIILFLVATATGAHS (SEQ ID NO:730)

Other embodiments are within the following claims:

What is claimed is:

1. A method for treating an angiogenesis-related disorder in a subject, the method comprising
administering a Tie1 ectodomain-binding agent and a vascular disrupting agent (VDA) to the subject.

2. The method of claim 1, wherein the Tie1 ectodomain-binding agent is administered for a period prior to the administration of the VDA.

3. The method of claim 1, wherein the Tie1 ectodomain-binding agent is administered following first administration of the VDA.

4. The method of claim 1, wherein the angiogenesis-related disorder comprises a neoplastic disease; an inflammatory disorder; an ocular angiogenic disease; Osler-Webber Syndrome; myocardial angiogenesis; plaque neovascularization; telangiectasia; hemophiliac joints; angiofibroma; or wound granulation.

5. The method of claim 1, wherein the subject is in need of reduced angiogenesis.

6. The method of claim 1, wherein the Tie1 ectodomain-binding agent increases Tie complex formation.

7. The method of claim 1, wherein the Tie1 ectodomain-binding agent increases tyrosine phosphorylation of Tie1.

8. The method of claim 1, wherein the Tie1 ectodomain-binding agent induces down modulation of Tie1 and Tie1/Tie2 complex from the surface of the cell.

9. The method of claim 1, wherein the Tie1 ectodomain-binding agent is an antibody.

10. The method of claim 9, wherein the antibody comprises at least one complementarity determining region (CDR) from E3 (DX-2240), E3b (DX-2220), M0044-A06; M0044-A11; M0044-B04; M0044-B05; M0044-B08; M0044-B09; M0044-B10; M0044-B12; M0044-C07; M0044-D01; M0044-E03; M0044-F03; M0044-F06; M0044-F09; M0044-G06; M0044-G07; M0044-G11; M0044-H03; M0044-H05; M0044-H07; M0044-H09; M0045-A02; M0045-A04; M0045-B01; M0045-B03; M0045-B11; M0045-C02; M0045-C11; M0045-C12; M0045-D01; M0045-D07; M0045-G01; M0045-G10; M0046-A1; M0046-B06; M0046-B10; M0046-G12; M0046-H03; M0046-H10; M0046-H11; M0047-B03; M0047-D01; M0047-D03; M0047-E10; M0047-G09; M0053-A02; M0053-A03; M0053-A05; M0053-A09; M0053-B09; M0053-B11; M0053-D03; M0053-D06; M0053-D12; M0053-E03; M0053-E04; M0053-E08; M0053-F04; M0053-F05; M0053-F06; M0053-F08; M0053-G04; M0053-G05; M0054-A08; M0054-B06; M0054-B08; M0054-C03; M0054-C07; M0054-E04; M0054-G01; M0054-G05; M0054-H10; M0055-A09; M0055-B11; M0055-B12; M0055-C05; M0055-C07; M0055-D03; M0055-D06; M0055-D12; M0055-E04; M0055-E06; M0055-E10; M0055-E12; M0055-F10; M0055-G02; M0055-G03; M0055-H04; M0056-A01; M0056-A06; M0056-B08; M0056-B09; M0056-C03; M0056-C04; M0056-E08; M0056-F01; M0056-F02; M0056-F10; M0056-F11; M0056-G03; M0056-G04; M0056-G08; M0056-G12; M0056-H04; M0056-H12; M0057-B05; M0057-H07; M0058-A09; M0058-D04; M0058-E09; M0058-F03; M0058-G03; M0058-H01; M0059-A02; M0059-A06; M0060-B02; M0060-H01; M0061-A03; M0061-C05; M0061-C06; M0061-F07; M0061-G12; M0061-H09; M0062-A12; M0062-B05; M0062-B07; M0062-C08; M0062-D04; M0062-E02; M0062-E03; M0062-E11; M0062-F10; M0062-G06; or M0062-H01.

11. The method of claim 1, wherein the method further comprises monitoring the subject.

12. The method of claim 11, wherein the monitoring is for one or more of: reduction in tumor size; reduction in a cancer marker; reduction in the appearance of a new lesion; reduction in the appearance of a new disease-related symptom; decrease of size of soft tissue mass; stabilization of size of soft tissue mass; or any parameter related to improvement in clinical outcome.

13. The method of claim 1, wherein the subject is a mammal.

14. The method of claim 13, wherein the mammal is a human.

15. A kit comprising
(a) a Tie1 ectodomain-binding agent;
(b) a VDA; and
(c) instructions for use in accordance with a method for treating an angiogenesis-related disorder in a subject.

16. A method for treating an angiogenesis-related disorder in a subject, the method comprising:
administering an effective amount of a Tie1 ectodomain-binding protein to a subject having an angiogenesis-related disorder, wherein said Tie1 ectodomain-binding protein is DX-2240; and
administering an effective amount of a vascular disrupting agent (VDA) to the subject, wherein said VDA is N-acetylcolchinol (5S)-5-(acetylamino)-9,10,11-trimethoxy-6,7-dihydro-5H-dibenzo[a,c]cyclohepten-3-yl dihydrogenphosphate (ZD6126).
